(19) United States
(12) Patent Application Publication
(10) Pub. No.: US 2020/0058215 A1
(43) Pub. Date: Feb. 20, 2020

(54) DETACHABLE INTRAVENOUS ALARM MONITORING SYSTEM

(71) Applicant: Matthew Ruggeri Novak, Winter Garden, FL (US)

(72) Inventor: Matthew Ruggeri Novak, Winter Garden, FL (US)

(21) Appl. No.: 16/661,523

(22) Filed: Oct. 23, 2019

Related U.S. Application Data
(63) Continuation-in-part of application No. 16/105,173, filed on Aug. 20, 2018, now abandoned.

(51) Int. Cl. G08B 25/10; A61M 5/142; G08B 21/18; G08B 5/36

(52) U.S. Cl. CPC G08B 25/10; A61M 5/142; G08B 21/18; A61M 2205/587; A61M 2205/18; A61M 2205/3561; A61M 2205/6054; G08B 5/36

(57) ABSTRACT

An IV pump remote alarm system is provided. The IV pump remote alarm system includes a modular unit detachably connected to a stationary unit, the stationary unit operative associated with an IV pump so that if an alarm signal is sent from the IV pump, the modular unit alerts a remote caregiver possessing the modular unit. Each modular unit provides visible and audio output devices for representing the alarm signal. The IV pump remote alarm system includes a charging unit adapted to charge a plurality of modular units, each modular unit associated with a different IV pump, wherein the charging unit could be located at a nursing station over 100 feet away from each IV pump and recuperating patient.

### CROSS-REFERENCE TO RELATED APPLICATION

This application claims the benefit of priority as a Continuation-in-Part of U.S. Non-provisional application Ser. No. 16/105,173, filed 20 Aug. 2018, the contents of which are herein incorporated by reference.

### BACKGROUND OF THE INVENTION

The present invention relates to medical devices and, more particularly, to an IV pump remote alarm system.

Hospital patients experience too many alarms or alerts while attempting to recuperate in their room. Specifically, IV pumps alarms, which frequently go off in the patient's hospital room, and can be very loud in order to get the attention of an attendant medical professional or caregiver. Unfortunately, the patient who is trying to rest and heal is subject to this frequent and annoying disturbance that it is unnecessary for the patient to experience since they are not capable of properly responding.

Furthermore, “alarm fatigue” is affecting today's healthcare workers more than ever: Call bells, telemetry alarms, fall-risk patients on bed alarms, overhead announcements, patients complaining, codes blue alert, IV pump alarms, and computer alerts, amongst others. As a direct cause, alarm desensitization has become increasingly more common amongst healthcare workers, leading to delayed reaction speeds to “true” alarms. In 2016, the Joint Commission made alarm management a National Patient Safety Goal.

Perhaps the biggest contributing factor to alarm fatigue is the IV pump alarm. The IV Pump is located in every patient room and usually “beeps” loudly 5-10× per day. Sometimes, the IV pump alarm will be so distracting that the patient himself is tired of listening to the alarm and will press the bedside “call bell” alarm to get the nurses attention, thus causing a second alarm for the same issue. If a registered nurse is busy caring of another patient, this IV alarm of the first patient may be beeping for as long as 15-30 min, sometimes hours. When a patient is very ill or delirious, this patient may start yelling, or worse, pull out the IV as a failed attempt to fix the IV alarm himself, thus worsening the problem. Needless to say, it costs resources to replace the IV, but most importantly, the agitated patient has removed their best option to receive immediate IV therapy. Additionally, the patient's caregivers' alarm fatigue has worsened.

The IV alarm noise itself is inherently disturbing; for instance, a family sitting at the bedside will become anxious from the noise. Doctors discussing the plan of care with the patient find this alarm very distracting when discussing a diagnosis or prognosis. The sound is disturbing to those working at the nursing station less than 50 feet away for those caregivers not directly involved in the patient's care. In yet other examples, the IV alarm may be contributing to lack of patient privacy. As the nursing station can be located sometimes over 50 ft from the patient rooms, the nurse may simply leave the patient's room door open in order to hear the IV beeping from the nursing station down the hall. This patient may prefer to keep their room door closed, but may be more concerned about receiving adequate care from their nurse and therefore not complain about having their privacy. This example is all too common in the acute hospitalized patient setting.

The IV pumps have alarms set to be activated if the IV pump deviates from set standards. The standards include but not limited to pressure, flow rate, total flow, flow interruption, gas in the infusion liquid, infusion complete, low IV pump battery, and the like. In other words, the IV alarm will beep for many reasons. When the pressure changes within the IV tubing, it causes the pressure sensor in the IV unit to detect a change in pressure and in response, trigger an alarm. Usually this is when a medication has completed. Other times, the IV will alarm when the plastic IV tubing has developed a “kink.” Kinks in the IV line can be due to misposition of the patient's arm during infusion—in fact, if the IV is located in the common antecubital fossa area (midpoint of the arm where patient bends his elbow), this IV tubing will “kink” anytime the patient bends his/her arm. Thus, the alarm beeps.

In response, the registered nurse must enter the patient room, turn off the alarm at bedside, check the IV site, and reset the pressure sensor and restart the infusion. If the patient is in a contact room, the nurse must gown up with appropriate contact precautions in order to prevent spread of infection (this is estimated around 30% of patients given high number of nursing home patients colonized with MRSA, ESBL, VRE, and other antibiotic resistant types of bacteria). If the IV tubing is “kinked” permanently, then the IV will need to be replaced. If the nurse has failed to place a new IV over the next few attempts, a special PICC team—a specialized team that places peripheral IV with the help of an ultrasound machine—will be ordered to place an IV line (PICC team needed in estimated up to 30-50% of elderly and obese patients during hospitalization). In this example, the IV pump may be as little as a false alarm or as important as requiring the PICC team. For this reason, it may be more useful in a hospital setting to use a system which uses a “silent alarm” or light indicator located at the nursing station for IV troubleshooting—a system which calls for any staff personnel, such as an IV pump specialist or PICC team member (not just the registered nurse) to address the “true” IV alarms on the floor in a more timely manner.

Alarm fatigue—Current IV pump alarms are noisy and often cause lack of sleep to the patient. Alarm fatigue also affects the healthcare worker in the hospital who must respond to multiple IV alarms, and distinguish between bed alarms, call bells, IV alarm, codes, and computer alerts, which often cause distraction when charting or caring for patients. Often times, IV pumps beep from the patient room and the nurse or caregiver must hunt down this alarm down the hall—leading to confusion and frustration, unsure if the alarm is his/her patient. This often leads to delayed response times to true alarms. Furthermore, if the caregiver is busy tending to another patient, this IV pump may continue beeping in the patient room for often 10-20 minutes at a time, distracting the patient from sleep and distracting nearby patients or even coworkers from charting, and delaying care to the patient.

The implementation of “smart IV pumps” could theoretically be utilized by coupling to the hospital computing network and electronic database to transmit data and alarms via the hospital network, however, the inventor has determined that such “smart IV pumps” involve complex and expensive systems and software.

As can be seen, there is a need for an IV pump remote alarm system enabling caretakers to perform their function without disturbing or alarming the patient hooked up to the IV pump. The IV pump embodied in the present invention has a detachable alarm system that can alert a caregiver or medical professional remotely at the nursing station. Whereby, the caregiver or medical professional is afforded the option to remove the IV pump alarm and carry it as a pager or place it at the nursing station in a rechargeable holder in order to allow more quiet time for the patient.

Furthermore, the present invention does NOT use a hospital network the Internet, and so does not incorporate complex expensive systems. Rather it uses a straight forward remote alarm system detachable from the main unit, enabling a proper, safe and quieter level of care.

By notifying appropriate personnel at the nursing station for true alarms, the present invention affords the patient timely responses and more sleep from less noise. Additionally, this silent alarms at the nursing station allow nurses to complete charting more efficiency with less distraction from false alarms, leading to more efficient workflow.

### The Present Invention Allows IV Pump Monitoring by Multiple Caregivers:

In current patient monitoring, one nurse or caregiver is assigned to each patient. Often times, due to staff shortages, this nurse is unable to attend to a beeping IV alarm or the nurse must cover for another nurse during lunch time or patient emergency. As it may be unpractical for that nurse in an emergency to hand off her personal tablets, phone, or computer to a different nurse (as in proposed “smartpump systems”), by setting up a central alarm and/or charging hub at the nursing station, a nurse can quickly tell if the alarm is his/her patient or if another nurse needs help. In this system, a patient's IV pump can be easily monitored by multiple caregivers, such nurses not directly assigned to the patient, or IV pump specialists who are specialized at placing IVs. This would help eliminating the wait time by the patient to receive IV medication or to place a new IV. Furthermore, placing a peripheral IV is very nurse dependent and can often times an unexperienced nurse can cause multiple failed needlesticks and unneeded trauma to the patient's arm when trying to place an IV. By assigning this task to an IV specialist, IV complication rates would decrease.

### The Present Invention Promotes Patient Privacy:

Currently, in most hospital systems, nurses or caregivers do not have any alert set up at the nursing station for IV pumps, not to mention a silent alerting system option. In the current systems, the caregiver must listen for a loud IV beeping noise from the patient room in order to attend to the IV pump. Unfortunately, most nurses will leave the patient room door open and turn the IV pump alarm up loudly in order to be audible from the nursing station, as some patient rooms can be located as far as 100 ft from the nursing station. Not only can this noise be more disturbing for the patient, family, nearby visitors, coworkers, etc., but the patient is now experiencing a breach in their privacy while their room door is open to bystanders, other patient visitors, or even employees not involved in the patient's direct care. The patient may forfeit this privacy, however, as they understand that it is more important to receive timely IV therapy by their nurse than to reveal their identity to visitors in the hospital. Unfortunately, in a time where digital social media is prevalent in society, especially teenagers, it is becoming increasingly important to promote patient privacy and implement new solutions when possible. It is estimate that approximately 30-75% of patient room doors are left open on the hospital ward during the day. This number decreases at night when patients are sleeping, however, and the number of complaints of being “unable to sleep because of the IV alarm” dramatically increases.

### The Present Invention is NOT Dependent on the Hospital Internet or New Electronic Software, and Therefore Less Susceptible to Online Hackers:

The IV pump in this system uses encrypted radiofrequency tags to send a ONE-WAY communication from the IV pump to the silent alarm at the nursing station. Therefore, a hacker could not change the setting of the IV medication infusion via wireless communication as this must be done manually using the stationary IV pump module. A similar analogy would be like a wireless car key sending a message to the specific car turn on the lights. In this analogy, the car key (like the stationary pump module) can only send a message to the car (detachable alarm unit) and does not receive any message from the car. For safety reasons, it is important to distinguish between the two types of wireless (one-way vs two-way communication) as a two-way system may be more susceptible to hackers than a one-way system. If the system were to use a hospital network, for example, this would indeed rely on a two-way system. If the hospital network were to become compromised, a hacker may be able to adjust the IV pump settings via the internet. In today's age, hospital security can be bypassed as easily as having an employee click on a virus URL link in their work email on their work tablet, work phone, etc. The imminent danger could be critical, and therefore, access to IV pumps settings should be limited to manual adjustment only and independent of a hospital network.

As a result, the system embodied in the present invention reduces alarm fatigue and allows for safer and more efficient workflow in a healthcare workplace by improving response rates to more critical or “true” alarms. By allowing an IV pump monitoring by multiple caregivers, true alarms can be answered by nurse managers, techs, doctors, or IV specialists, and not just the assigned nurse. Please see picture examples.

An advantageous element of the IV pump monitoring system is the plurality of detachable IV pump alarm units, each alarm unit assignable to one pump, patient and/or room number and where each alarm unit can be detachably associated with a central alarm and/or charging hub at a remote nursing station. Its alarm functions are easy to understand with only a light indicator and speaker, to receive one-way communication. Furthermore, it does not require adaptation or incorporation of new software into a hospital network. New hospital software usually requires extensive training and usually requires current caregivers to be “tech savvy.” This is a problem. Identifying as delays, glitches, viruses, red flags, computer alerts, and updating software can be time consuming, costly, and appears to be contributing more to alarm fatigue, delaying responses to true alarms, and increasing burnout rates.

Furthermore, the system embodied in the present invention promotes patient privacy. It is estimated that 30-75% of patient room doors are kept open in order to allow the nurse to listen for the IV pump alarm. Given the lack of options for patient IV pump monitoring, patient privacy will remain problematic for years to come unless a new system is adopted. Adopting “smartpumps” or new software technology within a hospital takes years or decades for hospitals to adopt due to cost and technical support. And such systems are impractical for most nonprofit hospital organizations due to the high cost.

One existing solution published by Scrivner US 2014/0337045 teaches of a system to monitor IV pump alarms at the nursing station by a caregiver using a personal tablet, however, this system pertains only for those in outpatient infusions with “smartpumps” to connect to the tablet via the hospital network. The outpatient infusion IV pump is monitored only by a single caregiver located in the same room as the patients. Moreover, Scrivner does not teach of monitoring critically ill hospitalized patients, or monitoring by multiple caregivers/specialists, nor is her monitoring performed by a caregiver outside the patient room. Scrivner does teach of the importance of reducing delay in IV therapy and reducing caregiver alarm confusion, as is often a problem with caring for multiple IV pumps. Scrivner also does not teach about a system which promotes increase response to true alarms, nor about protecting patient privacy. As with most hospitalized patients, these moments of privacy can be of utmost importance as images of patient illness (vomiting, bowel/bladder incontinence, yelling in pain, feeling disoriented, confused, becoming combative, etc.) can be quite disturbing to public visitors not involved in the patient care. In an era of digital social media, all measures should be taken to protect patient privacy in such situations.

### Arrangement and Relationships Between Systemic Components:

The IV pump unit may be situated in the patient room. Each IV pump may have its own detachable wireless alarm. The patient room is located in a separate room from the nursing station. Patient confidentiality and privacy is promoted using closed doors (unless patient is obtunded and must be monitored directly by staff). The IV pump alarm may be detached and placed in a central alarm and/or charging hub at the nursing station.

The IV pump alarm has silent mode option at nursing station which is most often utilized. Speaker audio is adjustable if needed for more critical IV infusions. The alarm unit only receives one-way wireless communication from stationary IV module (for safety reasons, this is not a two-way communication). The IV pump alarm is monitored by multiple caregivers to allow timely response to true alarms. The doctor, nurse, nursing manger, tech, or specialist, can visibly identify which patient IV pump is alarming (by light indicator) from the nursing station (without getting up and walking down the hall to “hunt down” the noise alarm). They can then troubleshoot the IV themselves or notify an IV specialist if the assigned nurse is busy.

In some embodiments, if the hospital administration were to create a separate area for the IV alarms, apart from the nursing station, then the detachable units could sit in a centralized hub in the IV specialist area, rather than or in addition to the nursing station, and there can be a plurality of central hubs. As long as the central hub is located outside the patient room and visible to multiple caregivers in order to promote the privacy of patients and enhance the efficiency of alarm responses by staff. This area typically must be located on the same hospital floor as IV pump, usually no further than 200 feet from IV pump in order to maintain radiofrequency connection.

In yet other embodiments, the alarm unit does not need to be physically detachable from the stationary unit. It may send radiofrequency transmission to the alarm receiver at the nursing station, so as long as to alert multiple caregivers at the nursing station.

The detachable IV pump must, in most cases, be made in conjunction with IV pump manufacturer and in compliance with OSHA and FDA safety standards. The encrypted radiofrequency tags must be specific to each pump so in that each stationary pump module can send one-way communication via radio transmitter to one alarm unit radio receiver. Furthermore, detachable alarm or alarm unit need a power source, such as a rechargeable battery.

This patient monitoring system is intended for the use and purposes of “inpatient” or continuous monitoring (usually 24+ hr. hospitalized patients), who require monitoring of IV alarms, especially by those trained in IV placement. The patient monitoring system is not intended for “outpatient” or elective procedures since such procedures do not require monitoring IV pumps outside a private patient room. As a disclaimer, if one chooses to use the IV pump with detachable alarm itself, in an outpatient setting for simple IV therapy, this pump may be used so without detaching alarm unit and function as an ordinary pump as described in FIG. 7.

### SUMMARY OF THE INVENTION

In one aspect of the present invention, an IV pump remote alarm system, includes a stationary unit configured to operatively associated with an IV pump control unit to relay at least one alarm signal therefrom; a modular unit removably and electrically couple to the stationary unit; at least one output device provided by the modular unit; and the modular unit configured to activate the at least one output device upon receiving the at least one alarm signal.

In another aspect of the present invention, the IV pump remote alarm system, includes one or more stationary units, each stationary unit configured to operatively associated with an IV pump control unit to relay at least one alarm signal therefrom; a wireless transmitter provided by each stationary unit; a female electrical port provided by each stationary unit; one or more modular units, each modular unit providing a wireless receiver configured to communicate with the wireless transmitter; a male electrical port provided by each modular unit; one or more visual output devices and one or more audio output devices provided by each modular unit; each modular unit configured to activate said output devices upon receiving the at least one alarm signal from an associated stationary unit; a charging stations providing a plurality of female electrical ports; and each female electrical port configured to removably and electrically couple to the male electrical port.

In yet another aspect of the present invention, a method of providing an intravenous alarm monitoring system, in an inpatient setting, includes the following: the setting having a plurality of discrete rooms and a remote station physically distanced from each of the discrete rooms; providing a stationary intravenous pump unit in each room, each stationary intravenous pump unit configured to activate a pump alarm if there is a deviation in an associated pump standard; providing an electronic component for sending only a one-way wireless communication of said pump alarm; providing a central hub in the remote station, the central hub configured to receive said one-way communication for each stationary intravenous pump unit, wherein each one-way communication is not dependent on an internet connection or software of the inpatient setting, wherein the electronic component comprises an encrypted radiofrequency tag, wherein the remote location includes a nursing station and an intravenous specialist station; further including at least one light-emitting output component provided by the central hub for each stationary intravenous pump unit, and operatively associating said at least one light-emitting output component to the respective one-way communication; further providing a modular unit for each stationary intravenous pump unit, wherein said modular unit detachably connects with said stationary intravenous pump unit and the central hub, and wherein each modular unit has a light-emitting output component operatively associated with the respective one-way communication, and wherein the central hub is at least 20 feet from the nearest discrete room.

These and other features, aspects and advantages of the present invention will become better understood with reference to the following drawings, description and claims.

### BRIEF DESCRIPTION OF THE DRAWINGS

FIG. 1 is a front elevational view of an exemplary embodiment of the present invention, shown in use;

FIG. 2 is a front perspective view of an exemplary embodiment of the present invention;

FIG. 3 is a side perspective view of an exemplary embodiment of the present invention;

FIG. 4 is an exploded perspective view of an exemplary embodiment of the present invention;

FIG. 5 is a perspective view of an exemplary embodiment of the present invention;

FIG. 6 is a perspective view of an exemplary embodiment of the present invention;

FIG. 7 is a top plan view of an exemplary embodiment of a typical hospital floor layout, illustrating the disadvantages of the prior art. Here, a nurse's station is often located at one side of the floor and patient rooms can be further down the hallway at the opposite end of the floor. This is typically done so that the nurse's station is encountered first by emergency personnel and visitors upon entering the floor, which also maintains a level or privacy for the patience being tucked away on the opposite end of the floor relative to such emergency personnel and visitors. Often, up to four IV pumps may be alarming at the same time, and since each nurse is responsible for a plurality of rooms/patients, nurses for these rooms with alarming IV pumps may be caring for another patient, taking a break, or ignoring the alarms by accident. Listening for audio IV alarms can be quite confusing to nurses sitting at the nursing station since pinpointing IV alarms to a certain room number can be difficult. Nurse A may be caring for rooms 17 and 16, while nurse B is caring for room 15, while nurse C is assigned to room 14. In this example, nurse A is ignoring the noise because she believes it is only alarming for room 15, when in fact, it is both 15 and 16. Nurse B is busy in another patient room and cannot attend to the alarm in room 15. Nurse C is unsure if room 14 is alarming, and wants to be sure, so she interrupts her workflow to check on the noise. Needless to say, the noise is fatiguing to patients and staff; and

FIG. 8 is a top plan view of an exemplary embodiment of a hospital floor layout incorporating the present invention, wherein the IV detachable alarm system is being used. These silent alarms are in a central hub near the nursing station with red light indicator. For this example, a total of three IV alarms are signaling, rooms 3, 15, and 16. Rooms 15 and 16 are on silent mode. Room 3 IV alarm is the only alarm audible, however, at a lower volume needed than in example of FIG. 7. This nurse has chosen to keep this alarm audible because it is a most critical IV therapy. No patients or staff are developing fatigue from the deafening noise. Alarms are numbered according to room and all nurses at the nursing station know exactly which IV pump requires attention. In this example, Nurse B has been assigned to bed 3 and 15. In this most unfortunate scenario, Nurse B was busy placing a new IV in room 15 when room 3 started to alarm. Fortunately, the charge nurse is aware of the “true alarm” in room 3 and has offered to help Nurse B. In this worst-case scenario, the time sensitive IV in 3 was corrected in time.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides an IV pump remote alarm system including a modular unit detachably connected to a stationary unit. The stationary unit associated with an IV pump so that if an alarm signal is sent from the IV pump the modular unit, providing visible and audio output device, will alert a remote caregiver in possession of the modular unit, even if that modular unit is over 100 feet away from the IV pump and recuperating patient.

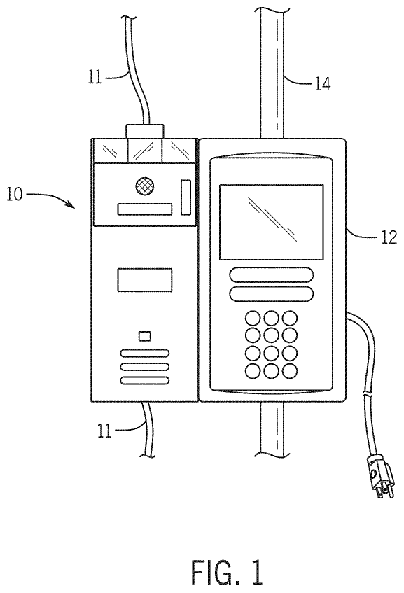

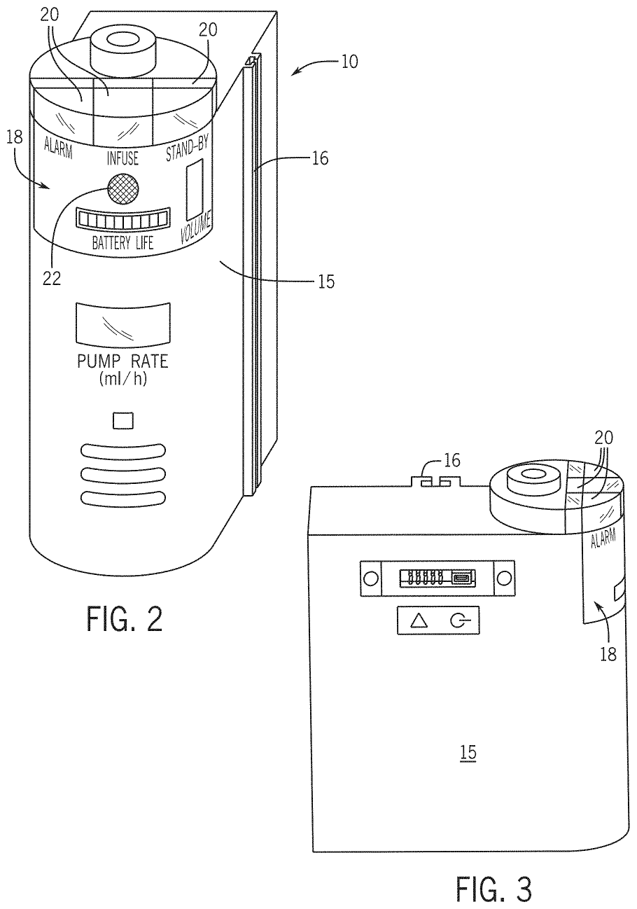

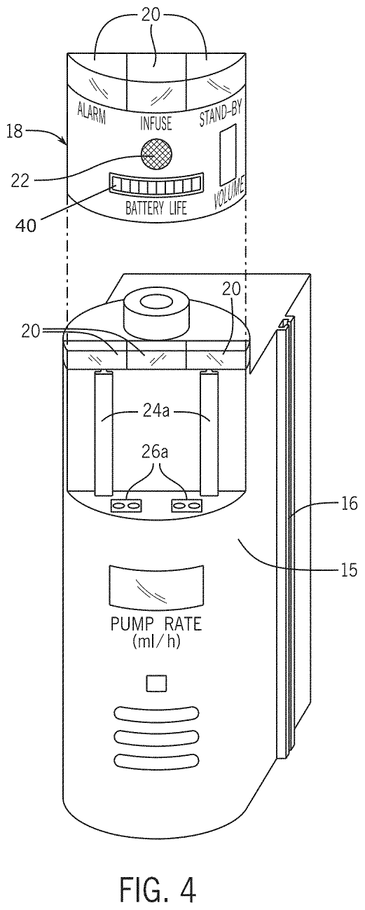

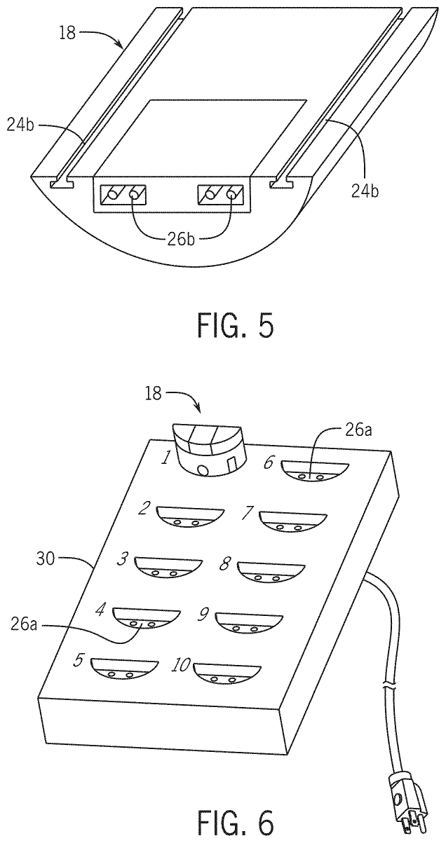

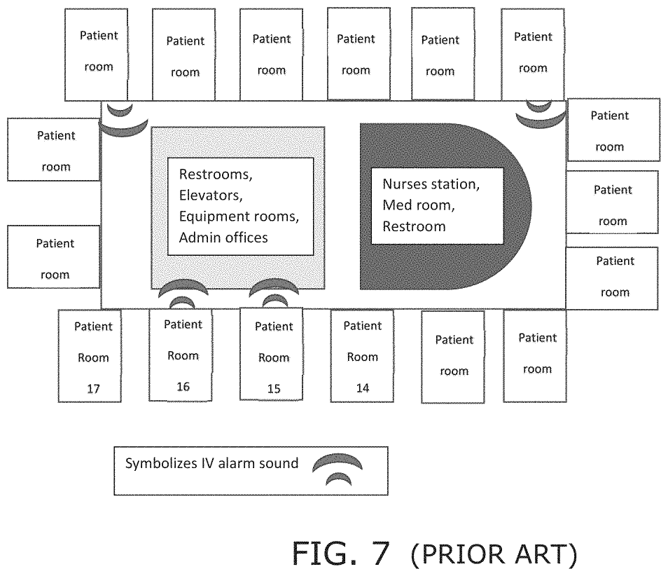

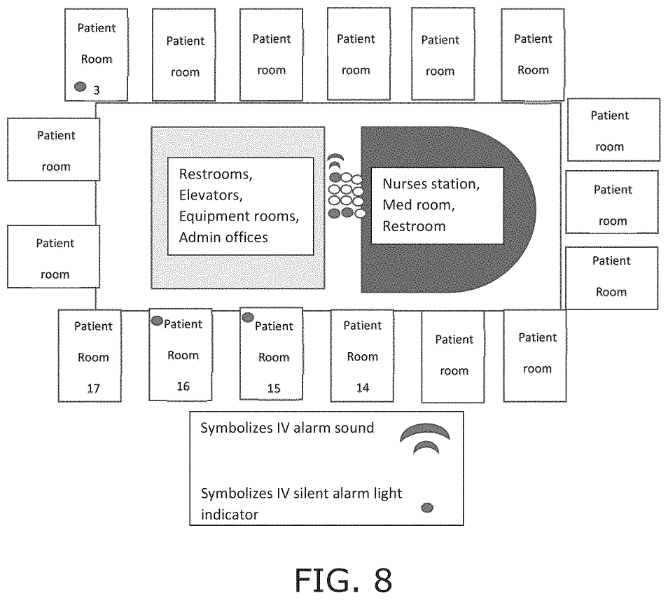

Referring to FIGS. 1 through 6, the present invention may include an IV pump remote alarm system 10. The pump remote alarm system 10 provides a modular unit 18 detachable from a stationary unit 15. The stationary unit 15 may be equipped to operatively associate with IV tubing 11 and removably attached to a control panel 12 along a standard IV pole 14, by way of a mounting rail 16 or the like.

The modular unit 18 is dimensioned and adapted to electrically couple to either the stationary unit 15 or a charging unit 30. As a result, the charging unit 30 may be substantially located apart from the stationary unit 15, such as a nursing station, while the stationary unit 15 would be located near the patient resting down on the other end of a hospital corridor. The stationary unit 15, modular unit 18 and charging unit 30 may have an intrinsic power sources (not shown). Each modular unit 18 may provide a set of male electric connectors 26b. The stationary unit 15 and the charging unit 30 may each provide a set of female electric connectors 26a (the charging unit 30 may provide a plurality of sets of female electric connectors 26a), wherein each set of female electric connectors 26a is adapted to electrically engage a set of male electric connectors 26b for transmitting power to the male electric connectors 26a. In other embodiments, that the charging unit 30 and the stationary unit 15 may provide “male” electric connectors 26b, while the modular unit 18 provides the corresponding “female” electric connectors 26a, it being understood that these corresponding electric ports/connectors 26a and 26b are adapted to electrically engage each other. Each modular unit 18 provides a battery indicator 40 telling the user when the modular unit 18 is fully charged and/or nearly out of charge.

The stationary unit 15 may provide a first connector 24a, typically adjacent to its set of female electric connectors 26a, while the modular unit 18 may provide a second connector 24b adapted to removably connect to the first connectors 24a. The first and second connectors 24a and 24b may be any mating connectors, such as tracks and grooves, or the like, that enable one object to removably attach to another object. One of the modular or stationary units 18 or 15 has may provide a release button to unlock and remove the modular unit 18 from the first connectors 24a. Once released, the detachable modular unit 18 will utilize power from an intrinsic power source.

Each modular unit 18 may include visual output devices 20, such a light source, and audio output devices 22, such as a speaker, to provide visible and audio alarms, respectively. The stationary unit 15 provides a remote wireless transmitter operatively associated with a respective wireless receiver of an associated modular unit 18. As a result, the visual and audio output devices 20 and 22 of the modular unit 18 may be operatively associated with the control panel 12, regardless of the former being detached from the stationary unit 15. During operation, therefore, the visual and audio output devices 20 and 22 of a remote modular unit 18 may be activated upon receiving an appropriate alarm signal; the control panel 12 being designed along the lines of traditional IV pump control circuitry: configured to transmit an alarm signal when certain predetermined thresholds are or are not being met. In certain embodiments, overall design of the stationary unit 15 (IV pump) is similar to the Alaris 8100 pump module, with similar color features of the alarm system which includes “Alarm” (red), “Infuse” (green), and “Standby” (yellow). In certain embodiments, the visual and audio output devices 20 and 22 may be operatively associated with the battery indicator 40 so as to further such an indication.

The charging unit 30 may be dimensioned and adapted to retain twelve or more detachable modular units 18. Each modular unit 18 has its own signal (or “pager number”) designed specifically for an associated stationary unit 15 (IV pump). This way, each alarm (modular unit 18) is designed specifically for an associated stationary unit 15/different discrete patients.

A method of using the present invention may include the following. The IV pump remote alarm system 10 disclosed above may be provided. The modular unit 18 can be detached from the main stationary unit 15 IV pump unit and stored in the charging unit 30. Typically, the charging unit 30 would be located at the nursing station, where the power source of each modular unit 18 can be recharged. The above-mentioned detachment of the modular unit 18 from the stationary unit 15 for up to 200 ft from IV pump so that a remote caregiver can be personally notified if necessary of the IV pump alarm signal because they can carry the modular unit 18 around with them instead of retain in the charging unit.

When the IV pump has completed its task or troubleshooting is indicated, and the alarm signal will be transmitted to the detachable alarm remote modular unit 18. The caretaker/medical professional can then silence the alarm by pressing the off switch on the alarm remote modular unit 18 and then assuming their task without the alarm disturbing the patient to whom the IV pump (and by the way the modular unit 18) is associated. The volume of the alarm can be adjusted with volume buttons on the alarm remote modular unit 18. The visual and/or audio output device 20 and 22 is adapted to deactivate once the task is completed on the main IV pump stationary unit 15.

Subsequent remote use, the modular unit 18 can be simply reattached to its main pump by clicking it into place, via the first and second connectors 24a and 24b, where it will resume power from the power supply of the stationary unit 15.

It should be understood, of course, that the foregoing relates to exemplary embodiments of the invention and that modifications may be made without departing from the spirit and scope of the invention as set forth in the following claims.

What is claimed is:

1. A method of providing an intravenous alarm monitoring system, in an inpatient setting, comprising:
the inpatient setting having a plurality of discrete rooms and a remote station physically distanced from each of the discrete rooms;
providing a stationary intravenous pump unit in each room, each stationary intravenous pump unit configured to activate a pump alarm if there is a deviation in an associated pump standard;
providing an electronic component for sending only a one-way wireless communication of said pump alarm; and
providing a central hub in the remote station, the central hub configured to receive said one-way communication for each stationary intravenous pump unit, wherein each one-way communication is not dependent on an internet connection or software of the inpatient setting.

2. The method of claim 1, wherein the electronic component comprises an encrypted radiofrequency tag.

3. The method of claim 1, wherein the remote location comprises a nursing station.

4. The method of claim 1, wherein the remote location comprises an intravenous specialist station.

5. The method of claim 1, wherein the remote location is both a nursing station and an intravenous specialist station.

6. The method of claim 1, further comprising at least one light-emitting output component provided by the central hub for each stationary intravenous pump unit, and operatively associating said at least one light-emitting output component to the respective one-way communication.

7. The method of claim 1, further providing a modular unit for each stationary intravenous pump unit, wherein said modular unit detachably connects with said stationary intravenous pump unit and the central hub, and wherein each modular unit has a light-emitting output component operatively associated with the respective one-way communication.

8. The method of claim 1, wherein the central hub is at least 20 feet from the nearest discrete room.
